(19) United States
(12) Patent Application Publication
(10) Pub. No.: US 2018/0301210 A1
(43) Pub. Date: Oct. 18, 2018

(54) EXPEDITED ADMISSIONS AND MEDICATION DELIVERY

(71) Applicant: CVS Pharmacy, Inc., Woonsocket, RI (US)

(72) Inventors: Christopher Hoff, Boston, MA (US); Melanie L. Kincer, Cincinnati, OH (US); Kulandhai Selvam Krishnaswamy, Levittown, PA (US); Daniel Long, Sellersville, PA (US); Brian Malachowski, Cranston, RI (US); Justin A. Reid, Providence, RI (US); Kelly D. Rennick, Warwick, RI (US); Steve Stecher, Huntington, IN (US); Thomas J. Weiss, Cherry Hill, NJ (US)

(21) Appl. No.: 15/951,463

(22) Filed: Apr. 12, 2018

Related U.S. Application Data
(60) Provisional application No. 62/484,885, filed on Apr. 12, 2017.

(51) Int. Cl. G16H 20/10; G16H 10/60

(52) U.S. Cl. CPC G16H 20/10; G16H 40/20; G16H 10/60

(57) ABSTRACT

A system, method, and computer program product for expedited admission and medication delivery provides for electronic transfer of continuity of care data, such as at least portions of continuity of care document including medication orders, when a patient is discharged from a first health care facility, such as an acute care facility. The continuity of care data is electronically transferred to a second health care facility, such as a non-acute care facility, to expedite admissions and medication delivery at the second health care facility.

### CROSS-REFERENCE TO RELATED APPLICATIONS

The present application claims priority to U.S. Patent Application No. 62/484,885, titled “Expedited Admissions and Medication Delivery,” filed Apr. 12, 2017, which is incorporated by reference in its entirety.

### BACKGROUND

The present disclosure relates to expedited patient admissions and medication delivery. In particular, the present disclosure relates to expedited admission and delivery of medications to patients being transferred from an acute care facility (e.g., a hospital providing acute care) to a non-acute care facility providing longer term health care (e.g., a skilled nursing facility).

Conventionally, when a patient is transferred from an acute care facility to a non-acute care facility, medical records and medication orders travel with the patient in the medical transport. A non-acute care facility generally does not have an in-facility pharmacy and in many cases may only have a limited supply of emergency medications onsite. On arrival at the non-acute care facility a nurse or other staff must review the charts and medication orders of a new patient, confirm the orders with the physicians, and enter the prescription orders into an ordering system or phone the orders in to a pharmacy.

The pharmacy typically receives the order (e.g., via fax, online system, telephone, etc.) from the non-acute care facility and enters the order into the pharmacy's systems. The pharmacy processes the order and sends the order out for delivery to the non-acute care facility. This transfer process is inefficient and can result in poor patient care and significant delays in time before a transferred patient receives their first dose of medication at the non-acute care facility. Some estimates suggest that the first 4 to 10 hours of patient transition is wasted gathering, transcribing, and implementing patient continuity of care documents.

### SUMMARY

The techniques introduced herein overcome the deficiencies and limitations of the prior art at least in part by providing a system and method for expediting admissions and medication delivery when a patient is discharged from a first health care facility and transferred to a second health care facility, where the first health care facility may be an acute care facility and the second health care facility may be a non-acute care facility.

In one embodiment, the present disclosure describes systems and methods for electronically communicating continuity of care data in a secure manner from an acute care facility to a non-acute care facility in response to the patient being discharged from the acute care facility. In one embodiment, the continuity of care data is a continuity of care document that includes medication orders for a discharged patient.

In one embodiment, additional auxiliary information is also generated from the medication orders of the continuity of care data and provided to the non-acute care facility. In one embodiment, this may include parsing the continuity of care data and processing it to generate lists of therapeutic equivalents or formulary equivalents for medications in the medication orders. In another embodiment, the continuity of care data may be processed to provide summary information or recommendations.

In one embodiment, a non-acute care facility receives the continuity of care data, reconciles medication orders in the continuity of care data, and provides instructions for fulfillment of the medication orders. In one embodiment, a determination may be made of available onsite medications relevant to the medication orders. In one embodiment, a pharmacy web portal includes a graphical user interface with an admitted patient dashboard to reconcile medication orders and provide instructions for fulfillment of the medication orders. In one embodiment, the medication orders in the continuity of care data are used to generate draft medication orders.

### BRIEF DESCRIPTION OF THE DRAWINGS

The present disclosure is illustrated by way of example, and not by way of limitation in the figures of the accompanying drawings in which like reference numerals are used to refer to similar elements.

FIG. 1 is a block diagram of an example system for expedited admissions and medication delivery in accordance with an embodiment.

FIG. 2A is a flowchart of an example method for expedited admissions and medication delivery in accordance with an embodiment.

FIG. 2B is a flow chart illustrating aspects of a more detailed method for expediting admissions and medication delivery in accordance with an embodiment.

FIG. 2C is a flow chart illustrating aspects of a method for expedited admissions and medication from the perspective of actions occurring outside of the non-acute care facility in accordance with an embodiment.

FIG. 3 is a block diagram of an example computing system for providing expedited admissions and medication delivery in accordance with an embodiment.

FIG. 4 is a block diagram of an example system for coordinating transfer of continuity of care data using an enterprise service system layer in accordance with an embodiment.

FIG. 5 illustrates an example of an admitted patient dashboard in accordance with an embodiment.

### DETAILED DESCRIPTION

With reference to the figures, reference numbers may be used to refer to components found in any of the figures, regardless whether those reference numbers are shown in the figure being described. Further, where a reference number includes a letter referring to one of multiple similar components (e.g., component 000a, 000b, and 000n), the reference number may be used without the letter to refer to one or all of the similar components.

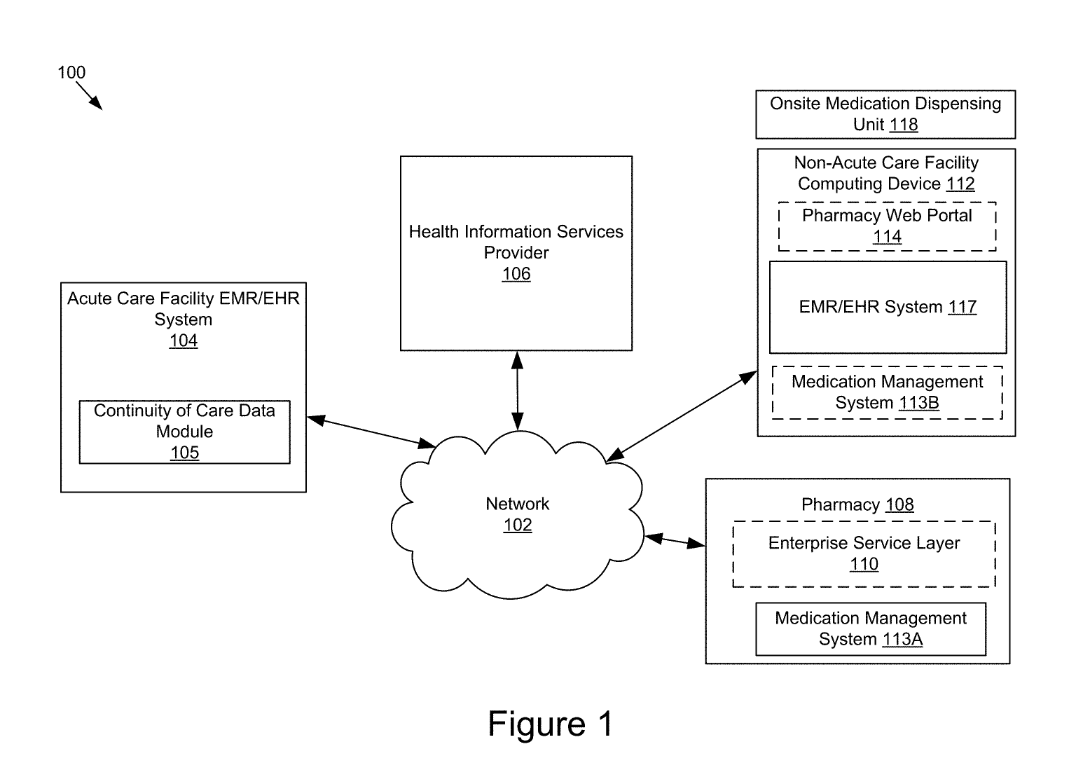

FIG. 1 is a block diagram of an example system 100 for expedited admissions and medication delivery in accordance with an embodiment. The illustrated system 100 may include an acute care facility electronic medical records (EMR) or electronic health records (EHR) system 104 associated with a first health care facility, which in one embodiment is an acute care health facility. In one embodiment, system 100 includes a health information services provider system 106, a pharmacy system 108 including an enterprise service layer 110, and a computing device 112 of a second health care facility, which in one embodiment is a non-acute care facility. The computing device 112 includes a pharmacy web portal 114. The computing device 112 may support or interface with an EMR/EHR system 117 of the non-acute care facility that is used to create patient profiles for new patients of the non-acute care facility. A network 102 provides for electronic communication to support interaction of the different components with one another, although other system configurations are possible including other devices, systems, and networks.

When a patient is discharged from the acute care facility, continuity of care data is generated in EMR/EHR system 104. In one embodiment, the continuity of care data is a continuity of care document that includes medication orders for the patient. More generally, the continuity of care document may include, for example, discharge orders, allergies, immunizations, discharge summaries, etc. The continuity of care document may also include other demographic information about the patient (e.g., age, sex, etc. about the patient).

In response to a patient being discharged in the records of the EMR/EHR system 104, the continuity of care data is released for secure electronic communication to other authorized entities in system 100. For example, when a decision is made to discharge a patient and transfer them to a non-acute care facility, the EMR/EHR system 104 may be updated to reflect the decision to discharge the patient and transfer them to a non-acute care facility. A continuity of care data module 105 may be implemented as computer program instructions in the EMR/EHR system 104 to support generating and releasing continuity of care data that is electronically communicated to the non-acute care facility via network 102. In one embodiment, the acute care facility is provided with address information to route the continuity of care data to the non-acute care facility. For example, the address information may include a unique provider identifier for the non-acute care facility.

In one embodiment, the entire continuity of care document is released for electronic communication and transported so that it can be used to provide the continuity of care document to the non-acute care facility. More generally in some embodiments only a relevant portion of the continuity of care document for expedited admissions and medications delivery may be electronically communicated, such as the medication orders or information derived from the medication orders.

It should be noted that the time at which a discharge occurs in the records of the EMR/EHR 104 may be at a slightly earlier point in time than when the patient physically leaves the grounds of the acute care facility, such as when the patient waits for transport to a non-acute care facility. In any case, the discharge in the medical records of the EMR/EHR 104 will typically occur much earlier in time than the physical arrival of a discharged patient at a non-acute care facility.

In some embodiments, an electronic inventory of medications available on-site at the non-acute care facility is available. For example, the computing device 112 and/or pharmacy web portal 114 may have access to an electronic inventory of medication available on-site at the non-acute care facility. For example, the non-acute care facility may have a medication storage unit or medication dispensing unit 118 managed by a medication management system, which is in one embodiment is a medication management system 113A of pharmacy 108 although alternatively a medication management system 113B could be part of the non-acute care facility. For example, information on the electronic inventory of medications could be provided to the pharmacy web portal 114 from the pharmacy 108 if the pharmacy maintains an electronic inventory of mediations at the non-acute care facility via medication management system 113A. In one embodiment, medication management system 113A has access to information on the electronic inventory of medications stored in medication dispensing unit 118 and provides this information to the non-acute care facility. In one embodiment, the availability of medications stored in medication dispensing unit 118 to fulfill part or all of the medication orders may be determined using drug identifiers to compare drug lists from the acute care facility with what is being dispensed by pharmacy 108 with what is stocked by medication dispensing unit 118. In some embodiments, the availability of a drug in the medication dispensing unit 118 is determined by comparing a drug code number of the drug being dispensed from the pharmacy 108 versus the drug code numbers of the drugs stored in the medication dispensing unit 118.

The network 102 may include any number of networks and/or network types. For example, the network 102 may include, but is not limited to, one or more local area networks (LANs), wide area networks (WANs) (e.g., the Internet), virtual private networks (VPNs), wireless wide area network (WWANs), WiMAX® networks, personal area networks (PANs) (e.g., Bluetooth® communication networks), various combinations thereof, etc. These private and/or public networks may have any number of configurations and/or topologies, and data may be transmitted via the networks using a variety of different communication protocols including, for example, various Internet layer, transport layer, or application layer protocols. For example, data may be transmitted via the networks using TCP/IP, UDP, TCP, HTTP, HTTPS, DASH, RTSP, RTP, RTCP, VOIP, FTP, WS, WAP, SMS, MMS, XMS, IMAP, SMTP, POP, WebDAV, or other known protocols.

The acute care facility EMR/EHR system 104 includes one or more computing devices having data processing and communication capabilities. The EMR/EHR also includes an associated memory for storing data records and computer program instructions. The computing devices may be configured to accurately store data relating to a patient and record the state of the patient over time. The EMR/EHR refers to everything typically found in a paper chart, such as medical history, diagnoses, medications, immunization dates, allergies. The EMR/EHR system 104 may be, for example, Apache cTAKES, AHLTA, athenaClinicals, Centricity EMR, Certify HealthLogix, Cerner EHR, CottageMed, COSTAR, Datix, DocNetwork, Dossia, EMIAS, EMIS Web, EpicCare EMR, FreeMED, GaiaEHR, GNUmed, GPASS, HOSxP, INPS Vision, Kareo EHR, MedcomSoft Record, MTBC WebEHR2.0, NHS Care Records Service, NHS Connecting for Health, OpenEMR, OpenMRS, Practice Fusion, SmartCare, Summary Care Record, TextID, TPP SystmOne, Vetter Software, VistA, VITAband, World Medical Card, ZEPRS, etc. The acute care facility EMR/EHR system 104 also includes a network interface (not shown) to communicate via network 102.

The health information services provider (HISP) system 106 corresponds to an organization that manages security and transport for health information exchange among health care entities or individuals. HISP system 106 may include an associated system or servers and network interfaces to support management of security and transport of health information.

In one embodiment, the acute care facility EMR/EHR system 104 communicates health record information, such as a document that includes at least relevant portions of medication orders, to other entities using the security and transport management supported by HISP system 106. In one embodiment, the continuity of care data, extracts of the continuity of care data, or data generated based on the continuity of care data may be transported to pharmacy 108 via an enterprise service layer. For example, the medication orders in the continuity of care data may be transported to pharmacy 108 via the enterprise service layer 110. However, as the non-acute care facility has a web connection with the pharmacy via the pharmacy web portal, continuity of care data transported to pharmacy 108 via the enterprise service layer 110 may be provided from the pharmacy to the computing device 112. However, in an alternate embodiment the continuity of care data may be directly transported to the non-acute care facility. In one embodiment, the HISP system 106 also provides security and transport management for continuity of care data electronically communicated from the acute care facility to the computing device 112 of the non-acute care facility.

In one embodiment, the continuity of care data is processed to generate a “playbook” that is transmitted to the non-acute care facility automatically to provide a summary of key information to aid staff at the non-acute care facility. As an example, the summary may include portions of the medication orders, a summary of the acute encounter, demographics, clinical summaries, allergies, etc. For example, in some embodiments, the medication orders may be parsed to generate a summary of medications in the medication orders. Additionally, in some embodiments additional information for staff at the non-acute care facility may be generated in regards to how to handle various medication issues associated with the medication orders. For example, the medication orders in a continuity of care document may be parsed to generate a sequence of medications. Additional information for the medications may also be optionally generated to assist nursing staff fulfill the medication orders. Moreover, processing may be performed to identify therapeutic alternatives to individual medications consistent with the patient's profile such as patient allergies and other information in the continuity of care data. Similarly, formulary equivalents may also be identified consistent with the continuity of care data. The identification of therapeutic alternative and formulary equivalents may be performed for a variety of objectives, including identifying preferred versions of drugs to avoid delays at the pharmacy (including avoiding any billing delay). The generation of the playbook and therapeutic alternatives may be performed in different portions of the system 100 depending on implementation details.

In one embodiment, the non-acute care facility computing device 112 is a computing device or computing system having data processing and communication capabilities, including a network interface. While a single computing device 112 is illustrated, more generally it would be understood that the components and functionality of computing device 112 could be distributed and implemented in different devices, systems, servers, and services.

In one embodiment, the computing device 112 runs or accesses a pharmacy web portal 114. In one embodiment, the pharmacy web portal is provided by the pharmacy 108. In some embodiments, the pharmacy web portal 114 includes an admitted patient dashboard (not shown) that provides visibility for non-acute care facility staff (e.g., a nurse) to medication status for new admissions. For example, the dashboard may provide a proactive alert of issues or information needed to process medication orders. Further, the dashboard may provide notification of medications available onsite at the non-acute care facility via an onsite medication dispensing unit 118. The medication management system of the non-acute care facility may have access to an electronic inventory of onsite medication. For example, in one embodiment, the onsite medication dispensing unit 118 has an electronic inventory status that can be read, directly or indirectly, by computing device 112 or the pharmacy web portal 114 to determine the status of medications currently available in the onsite medication dispensing unit 118. For example, the computing device 112 could directly read the electronic inventory status. Alternatively, a pharmacy 108 having access to the electronic inventory of the onsite medication dispensing unit 118 could provide this information to the computing device 112. In one embodiment, the dashboard may provide a delivery schedule for medications that will be fulfilled by the pharmacy.

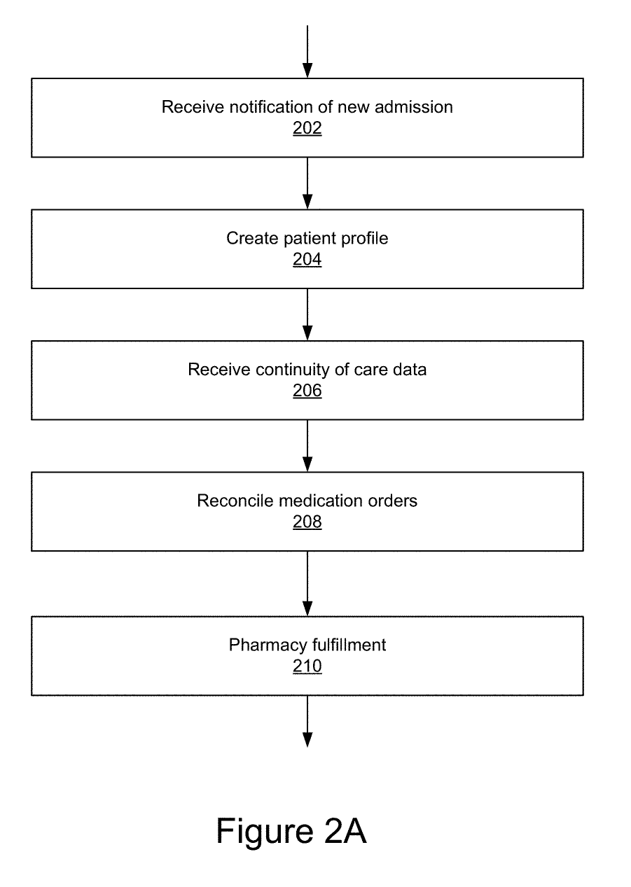

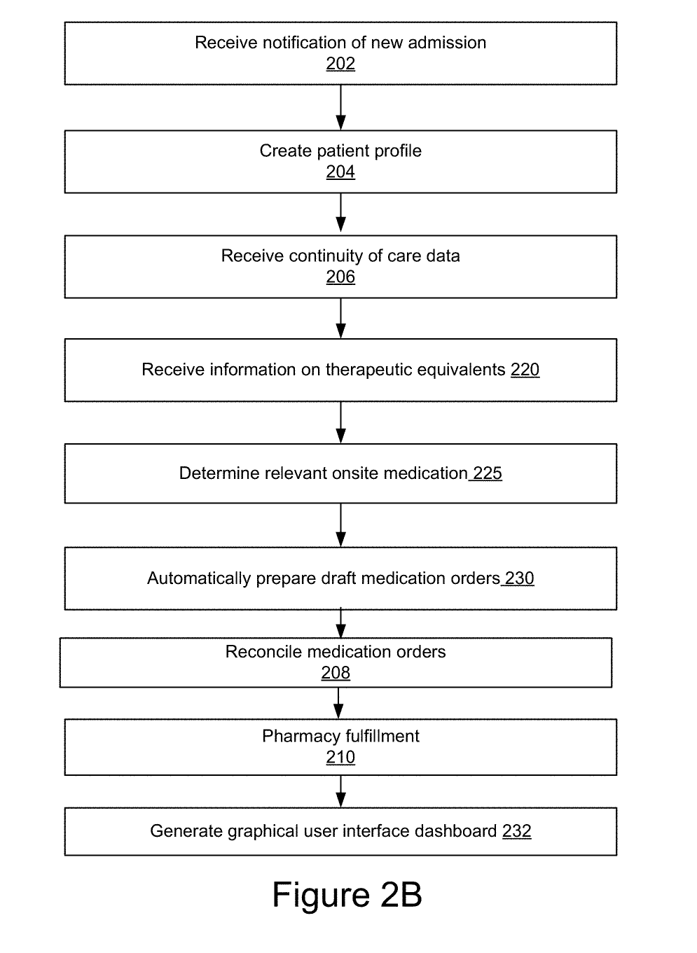

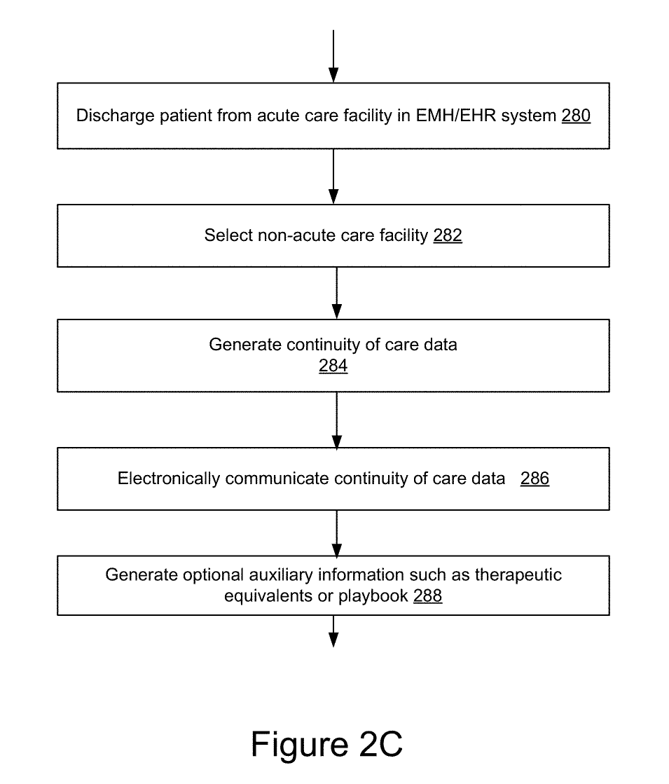

FIG. 2A is a flowchart of an example method for expedited admissions and medication delivery. At block 202, a non-acute care facility receives notification of a new admission from a transferring acute care facility. In some embodiments, this notification is in response to the discharge of a patient from the EMR/EHR system 104 of the acute care facility. This notification may be provided via an electronic notification, by fax, or other means and provided to EMR/EHR system 117 of the non-acute care facility.

In one embodiment, at block 204 a patient profile is created in the non-acute care facility, such as a patient profile in EMR/EHR system 117. In some embodiments, when a patient profile is created, the patient profile is transmitted to the pharmacy 108 to expedite order fulfillment. In some embodiments, pharmacy 108 also receives a continuity of care document from the acute care facility (e.g., via the health information services provider system 106 and the enterprise service layer 110) or relevant portions thereof to aid a pharmacy 108 to be prepared to service orders.

At block 206, the non-acute care facility directly or indirectly receives continuity of care data electronically transmitted from the acute care facility. In one embodiment, the enterprise service layer 110 of pharmacy 108 receives the continuity of care data, which is then provided to the non-acute care facility's computing device 112. Depending on implementation details, the continuity of care data may alternatively be directly received by the non-acute care facility's computing device 112 or by specific components thereof such as the medication management system 113B. In one embodiment, the entire continuity of care document is received. However, more generally extracts of the continuity of care document may be received, such as the medication orders or information parsed or otherwise derived from the medication orders.

At block 208, the non-acute care facility staff reconciles medication orders for the patient. One aspect of reconciling medication orders is that the continuity of care data may be used in some embodiments to automatically create draft medication orders. In some embodiments, the continuity of care data that is received is used to automatically create draft medication orders. For example, in one embodiment a message is sent to the non-acute care facility computing device 112 that includes a flag or field to indicate to a receiving interface of non-acute care facility computing device 112 that a message should be treated by EMR/EHR system 117 as a draft medication order. For example, the HL7 messaging standard includes an optional order control reason field that may be populated with a suggested order flag to inform EMR/EHR system 117 that the message is not a finalized pharmacy order but should be displayed by EMR/EHR system 117 as a draft order. In some embodiments, the draft medication orders are then provided to nurses and doctors for review and confirmation. In some embodiments, another aspect of reconciling medication orders may include determining the availability of relevant onsite inventory, such as inventory in an onsite medication dispensing unit 118, to satisfy part or all of the medication orders from onsite medication stores at the non-acute care facility or via therapeutic equivalents for formulary equivalents.

In some embodiments, the pharmacy web portal 114 notifies the non-acute care facility staff that at least a portion of the medication orders can be fulfilled from an onsite medication storage system or a medication dispensing unit 118. In other embodiments, the pharmacy 108 fulfills at least a portion of the medication orders via delivery from a pharmacy facility.

At block 210, the medication orders are fulfilled by the pharmacy 108. Status alerts may also be provided by the pharmacy regarding expected delivery time. Other types of alerts may also be generated, such as error alerts if there are problems ordering a medication or issues that need to be resolved to fulfill an order.

FIG. 2B illustrates in more detail some optional additional aspects of the flow chart of FIG. 2A. At block 206, the continuity of care data is received. At block 220 information on therapeutic equivalents is received, which may be by secure electronic communication, fax, or by other means. At block 225, a determination is made of relevant onsite medications or therapeutic equivalents capable of fulfilling at least a portion of the medication orders. At block 230, draft medication orders may optionally be generated for review of staff. The draft medication orders may, for example, be automatically created based on the medication orders and further include information on any onsite medications or therapeutic equivalents to aid staff in reconciling medication orders at block 208 for pharmacy fulfillment at block 210. As illustrated in block 232, in some embodiments a graphical user interface is generated, such as a dashboard in the pharmacy portal, to display information on the medication orders, the availability of onsite medications relevant to the medication orders (e.g., directly satisfying a portion of the medication orders or as therapeutic equivalents), and to aid in reconciling medication orders for pharmacy fulfillment.

FIG. 2C illustrates a method from the perspective of the acute care facility. At block 280, the patient is discharged in the EMR/EHR system 104 of the acute care facility.

In one embodiment, as part of the discharge process, a selection is made at block 282 of a non-acute care facility the patient is to be transferred to.

As part of the discharge process, continuity of care data is generated or otherwise released at block 284 for the discharged patient, where the continuity of care data includes medication orders and may be in the form of a continuity of care document.

At least a portion of the continuity of care data, such as at least a portion of the medication orders, is electronically communicated in block 286 to the selected non-acute care facility. This may include an entire continuity of care document, the medication orders, or information derived from the continuity of care document. The electronic communication may be implemented in a secure manner via the network in accordance with the security provisions of the HISP system. It will also be understood that this communication may be performed through intermediate layers, processes, or systems in system 100.

Additional optional information may be generated from the continuity of care data, such as a playbook or a listing of therapeutic equivalents in block 288, which may be performed by one or more portions of the system 100, depending on implementation details.

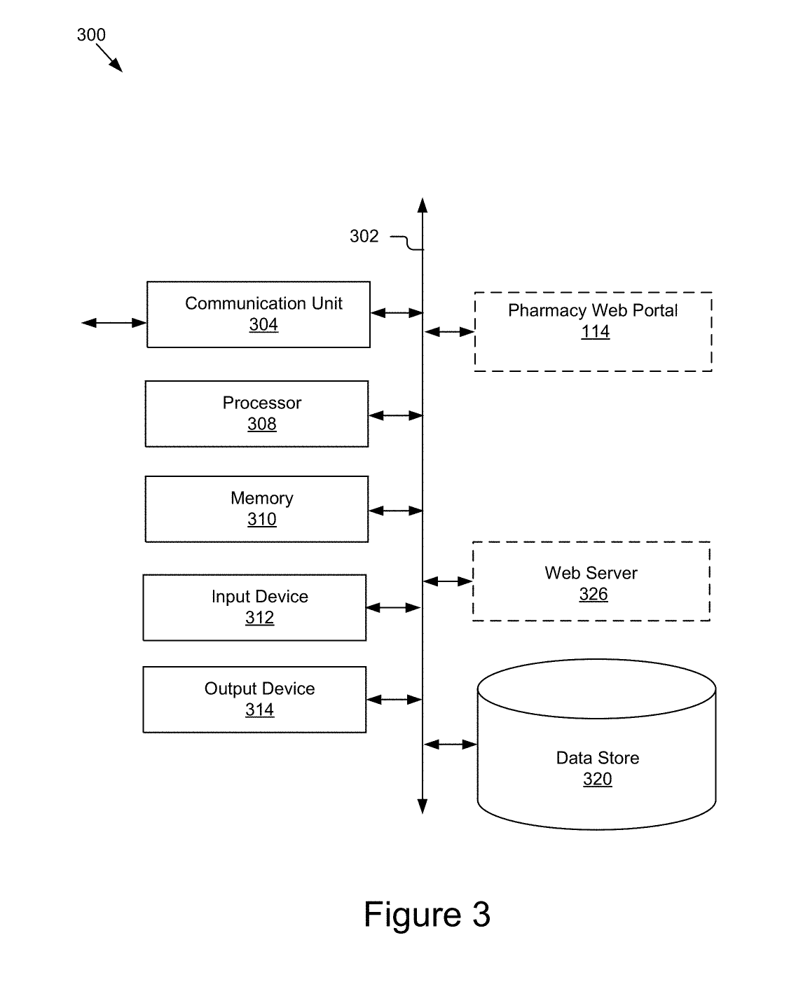

FIG. 3 is a block diagram of an example computing system 300, which may represent the computer architecture of a computing device, for example an acute care facility EMR/EHR system 104, a pharmacy computing system, or a non-acute care facility computing device 112, as depicted in FIG. 1, depending on the implementation.

As depicted in the example of FIG. 3, the computing system 300 may include a web server 326 and/or a pharmacy web portal 114, depending on the configuration.

The web server 326 includes computer logic executable by the processor 308 to manage content requests. The web server 326 may include an HTTP server, a REST (representational state transfer) service, or another suitable server type. The web server 326 may receive content requests (e.g., object search requests, HTTP requests) from computing devices, such as non-acute care facility computing device 112, to provide the pharmacy web portal 114 and/or provide content for the pharmacy web portal 114, although other configurations are also possible and contemplated.

In some instances, the web server 326 may format the content using a web language and provide the pharmacy web portal content to a corresponding application or web browser (not shown) for processing and/or rendering to the user for display. The web server 326 may be coupled to the data store 320 to store retrieve, and/or manipulate data stored therein and may be coupled to the pharmacy 108 to facilitate their operations. For example, the web server 326 may allow a user on a non-acute care facility computing device 112 to communicate with the pharmacy 108 and/or the other components of the system 100.

In one embodiment, the pharmacy web portal 114 includes computer logic executable by the processor 308 to manage and orchestrate fulfillment of prescription orders for patients admitted to the non-acute care facility, as discussed elsewhere herein. The pharmacy web portal 114 may be coupled to the data store 320 to store, retrieve, and/or manipulate data stored therein and may be coupled to the web server 326 and/or other components of the system 100 to exchange information therewith.

As depicted, the computing system 300 may include a processor 308, a memory 310, a communication unit 304, an output device 314, an input device 312, and a data store 320, which may be communicatively coupled by a communication bus 302. The computing system 300 depicted in FIG. 3 is provided by way of example and it should be understood that it may take other forms and include additional or fewer components without departing from the scope of the present disclosure. For instance, various components of the computing device may be coupled for communication using a variety of communication protocols and/or technologies including, for instance, communication buses, software communication mechanisms, computer networks, etc. While not shown, the computing system 300 may include various operating systems, sensors, additional processors, and other physical configurations. The processor 308, memory 310, communication unit 304, etc., are representative of one or more of these components.

The processor 308 may execute software instructions by performing various input, logical, and/or mathematical operations. The processor 308 may have various computing architectures to method data signals (e.g., CISC, RISC, etc.). The processor 308 may be physical and/or virtual and may include a single core or plurality of processing units and/or cores. In some implementations, the processor 308 may be coupled to the memory 310 via the bus 302 to access data and instructions therefrom and store data therein. The bus 302 may couple the processor 308 to the other components of the computing system 300 including, for example, the memory 310, the communication unit 304, the input device 312, the output device 314, and the data store 320.

The memory 310 may store and provide access to data to the other components of the computing system 300. The memory 310 may be included in a single computing device or a plurality of computing devices. In some implementations, the memory 310 may store instructions and/or data that may be executed by the processor 308. For example, the memory 310 may store one or more of the web server 326, pharmacy web portal 114, and their respective components, depending on the configuration. The memory 310 is also capable of storing other instructions and data, including, for example, an operating system, hardware drivers, other software applications, databases, etc. The memory 310 may be coupled to the bus 302 for communication with the processor 308 and the other components of computing system 300.

The memory 310 may include a non-transitory computer-usable (e.g., readable, writeable, etc.) medium, which can be any non-transitory apparatus or device that can contain, store, communicate, propagate or transport instructions, data, computer programs, software, code, routines, etc., for processing by or in connection with the processor 308. The memory 310 may include computer program instructions for implementing methods of expedited admissions and medication delivery as described above. In some implementations, the memory 310 may include one or more of volatile memory and non-volatile memory (e.g., RAM, ROM, hard disk, optical disk, etc.). It should be understood that the memory 310 may be a single device or may include multiple types of devices and configurations.

The bus 302 can include a communication bus for transferring data between components of a computing device or between computing devices, a network bus system including the network 102 or portions thereof, a processor mesh, a combination thereof, etc. In some implementations, the web server 326, pharmacy web portal 114, and various other components operating on the computing system 300 (operating systems, device drivers, etc.) may cooperate and communicate via a communication mechanism included in or implemented in association with the bus 302. The software communication mechanism can include and/or facilitate, for example, inter-method communication, local function or procedure calls, remote procedure calls, an object broker (e.g., CORBA), direct socket communication (e.g., TCP/IP sockets) among software modules, UDP broadcasts and receipts, HTTP connections, etc. Further, any or all of the communication could be secure (e.g., SSH, HTTPS, etc.).

The communication unit 304 may include one or more interface devices (I/F) for wired and wireless connectivity among the components of the system 100. For instance, the communication unit 304 may include, but is not limited to, various types known connectivity and interface options. The communication unit 304 may be coupled to the other components of the computing system 300 via the bus 302. The communication unit 304 can provide other connections to the network 102 and to other entities of the system 100 using various standard communication protocols.

The input device 312 may include any device for inputting information into the computing system 300. In some implementations, the input device 312 may include one or more peripheral devices. For example, the input device 312 may include a keyboard, a pointing device, microphone, an image/video capture device (e.g., camera), a touch-screen display integrated with the output device 314, etc. The output device 314 may be any device capable of outputting information from the computing system 300. The output device 314 may include one or more of a display (LCD, OLED, etc.), a printer, a haptic device, an audio reproduction device, a touch-screen display, a remote computing device, etc. In some implementations, the output device is a display which may display electronic images and data output by a processor of the computing system 300 for presentation to a user, such as the processor 308 or another dedicated processor.

The data store 320 may include information sources for storing and providing access to data. In some implementations, the data store 320 may store data associated with a database management system (DBMS) operable on the computing system 300. For example, the DBMS could include a structured query language (SQL) DBMS, a NoSQL DMBS, various combinations thereof, etc. In some instances, the DBMS may store data in multi-dimensional tables comprised of rows and columns, and manipulate, (e.g., insert, query, update and/or delete), rows of data using programmatic operations.

The data stored by the data store 320 may be organized and queried using various criteria including any type of data stored by them, such as a user identifier, a prescription record identifier, user attributes, pharmacy item attributes, retail item attributes, store identification, location data, etc. The data store 320 may include data tables, databases, or other organized collections of data.

The data store 320 may be included in the computing system 300 or in another computing system and/or storage system distinct from but coupled to or accessible by the computing system 300. The data stores 320 can include one or more non-transitory computer-readable mediums for storing the data. In some implementations, the data stores 320 may be incorporated with the memory 310 or may be distinct therefrom.

The components 304, 308, 310, 312, 314, 326, and/or 114 may be communicatively coupled by the bus 302 and/or the processor 308 to one another and/or the other components of the computing system 300. In some implementations, the components 304, 308, 310, 312, 314, 326, and/or 114 may include computer logic (e.g., software logic, hardware logic, etc.) executable by the processor 308 to provide their acts and/or functionality. In any of the foregoing implementations, these components 304, 308, 310, 312, 314, 326, and/or 114 may be adapted for cooperation and communication with the processor 308 and the other components of the computing system 300.

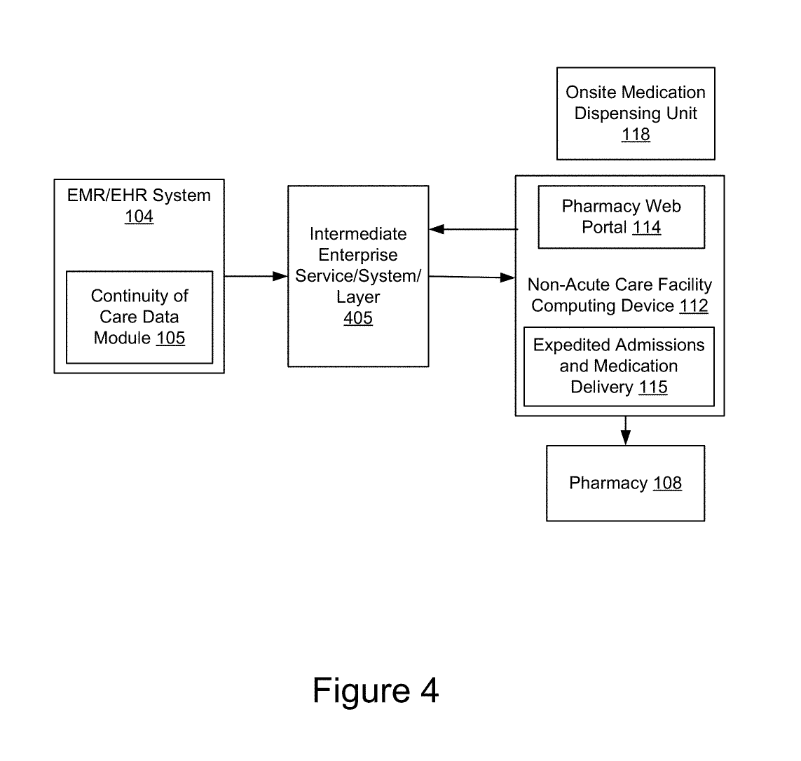

FIG. 4 illustrates another example of a system. In some embodiments, an enterprise service system 405 supports an enterprise service layer. That is, the enterprise service system 405 may be implemented as a centralized system supporting an enterprise service layer 110 as part of a larger system that includes a local regional pharmacy 108. For example, continuity of care data from an acute care facility 104 may be provided to the enterprise service system 405. The enterprise service system 405 may also perform processing of continuity of care data such as generation of playbooks, therapeutic equivalents, and preparation of draft medication orders. In one embodiment, an enterprise service system 405 provides an enterprise service layer that receives continuity of care data sent from the EMR/EHR system 104 of the acute care facility when a patient is discharged. In some embodiments, the enterprise service system 405 may temporarily hold the data in a data store, pending the generation of a patient profile at the computing device 112. As one example, a patient profile may be generated in response to a notice of a new patient admission that may be prompted by receiving notification of a patient to be transferred to the non-acute care facility. In one embodiment, the non-acute care facility first creates a new patient profile and then a request for the continuity of care data from the enterprise service system 405 is triggered. For example, the non-acute care facility may receive a notice of a new patient admission, create a patient profile, and then in response a request for the continuity of care data is triggered. However, more generally, enterprise service system 405 could also implement an enterprise service layer to automatically forward the continuity of care data to the non-acute care facility.

Additionally, in some embodiments, the enterprise service system 405 may generate playbooks and a listing of therapeutic equivalents, which could be communicated electronically, via fax, or by other means to the non-acute care facility and the computing device 112.

In some embodiments, the enterprise service system 405 utilizes the HISP system 106 connectivity and security features to communicate with the acute care facility. In one embodiment, communication between the acute care facility and the intermediate enterprise service system 405 is performed via a healthcare information service provider, such as SureScripts™. The enterprise service system 405 may also include firewalls or other security features to protect patient data received from the acute care facility. The enterprise service system 405 may also include directory information to aid in communicating between an acute care facility and a non-acute care facility. In one embodiment, the enterprise service system 405 also implements an enterprise service layer to securely communicate with and coordinate actions with other components of the system 100 such as the pharmacy 108. In some embodiments, the non-acute care facility computing device 112 or the pharmacy web portal 114 includes one or more features to support interactions with the enterprise service system 405. For example, an expedited admissions and medication delivery module 115 may comprise computer program instructions stored on a computer storage medium and executable by a processor to coordinate communications and interactions with the enterprise service system 405 and other components to, for example, receive the continuity of care data in computing device 112 in order to support expediting admissions and reconcile medication orders.

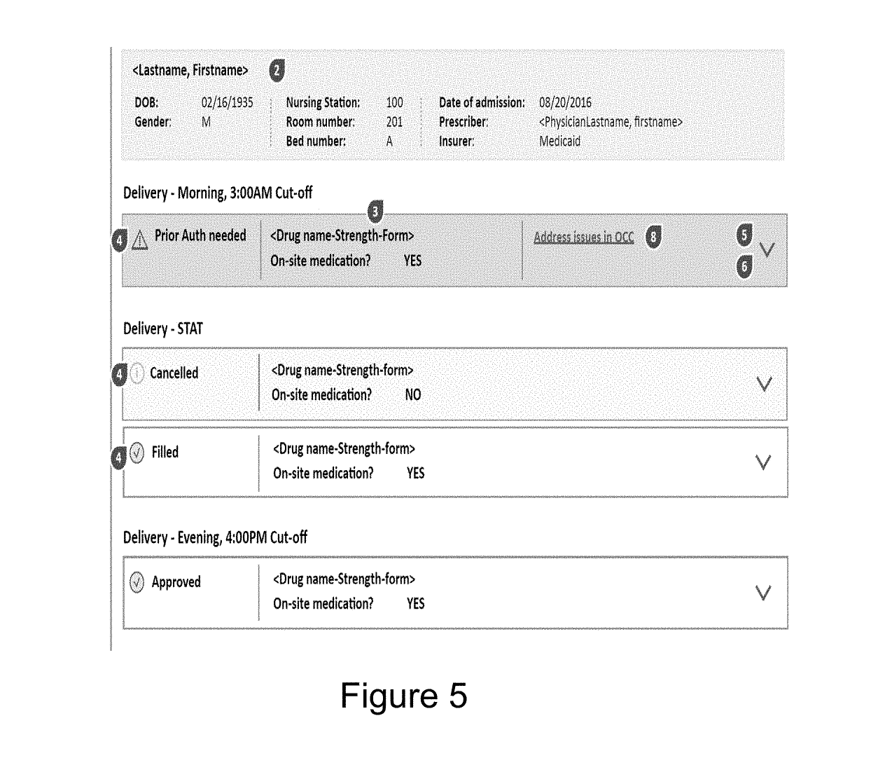

As previously discussed, the pharmacy web portal 114 may implement an admitted patient dashboard that may, for example, be displayed on a computer display at the non-acute care facility. FIG. 5 illustrates an example of an admitted patient dashboard in accordance with an embodiment. The admitted patient dashboard may include features to aid in reconciling medication orders and providing instructions for fulfilling medication orders. In one embodiment, an admission dashboard may provide visibility to medication orders for newly admitted patients. In one embodiment, it may provide notification of medications available onsite, provide proactive alerts of issues needed to process medication orders, and provide a status and delivery schedules for medications that will be fulfilled by the pharmacy. The dashboard may also include an indication of rush (stat) deliveries. In some embodiments, the admitted patient dashboard also includes features for providing instructions to a pharmacy to fulfill medication orders. More generally, the admitted patient dashboard may take different forms than that illustrated in FIG. 5 and include different features, depending on implementation details, to aid staff at a non-acute care facility to expedite admissions and medication delivery after the continuity of care data has been received.

As set forth in detail above, the technology described herein provides an innovative approach to the discharge process for discharging a patient from a first health care facility (e.g., an acute care facility) and transferring them to a second health care facility (e.g., a non-acute care facility). For example, in one embodiment, the technology described herein jump-starts admissions and pharmacy operations with receipt of data from the acute care facility at the time of discharge, minimizes order entry and administrative activities for nursing staff at the non-acute care facility receiving the transfer, reduces risk of transcription and other data entry errors, and helps reduce time to a first dose for selected medications through use of onsite medications.

In some embodiments, a determination can be made of medications on site to fulfill the medication orders as well the availability of any therapeutic alternatives. Any additional necessary medications can be ordered and the delivery status confirmed. This provides many potential benefits. In some cases, it may reduce the time that patients wait for medication, thus improving patient care, comfort, and safety. Also, it may in some cases reduce charges associated for rush (stat) delivery of medications. Additionally, it provides other potential improvements in convenience and safety by reducing the need for nurses at the non-acute care health facility to transcribe the continuity of care data, reducing labor and also helping to prevent transcription errors.

Reducing the time to first dose of medication improves patient care. When new patients arrive at a non-acute care facility, they generally do not have any of their required medications with them and the non-acute care facility may only have small supplies of emergency medications on hand to stabilize their condition and relieve pain. For example, a non-acute care facility may have an onsite medication dispensing unit 118 stocked at any one time with only a limited supply of medications.

A non-acute care facility typically depends on a pharmacy to deliver the patient's medications in a timely fashion or else risks the health of the patient, possibly requiring a return to the hospital. Therefore, non-acute care facilities are sensitive to medication availability and timeliness of admission orders. The techniques introduced herein allows non-acute care facilities to track the arrival of medications, see and resolve issues with the orders, and quickly determine which orders can be obtained from their onsite medication dispensing units 118 for immediate use.

Further, upon admission, urgent medications must either be obtained from an in-facility source or delivered via a special urgent delivery at the pharmacy. In one embodiment, the techniques introduced herein will help non-acute care facilities to quickly view which admissions orders can be obtained via their onsite medication dispensing unit 118 for immediate use. This functionality may reduce the need for stat deliveries which are more expensive, due to courier charges, and have a negative impact on overall pharmacy operations by causing staff to interrupt their normal work to address the urgent medication first.

In one embodiment, the techniques disclosed herein provides for expedited admissions and medication delivery. In one embodiment, an admitted patient dashboard is provided for visibility and alerts regarding new patient prescription order availability. In one embodiment, the admitted patient dashboard provides visibility to the status of orders throughout the fulfillment and delivery workflow for new admitted patients. In one embodiment, the admitted patient dashboard further creates alerts for orders that fall into an error status, such as billing related issues. Additionally, in one embodiment techniques disclosed herein include displaying alerts on the admitted patient dashboard as well as providing alternative delivery methods for alerts (e.g., via email or SMS). In one embodiment, for orders that have errors, the admitted patient dashboard provides a link from the dashboard to a pharmacy connection that allows the non-acute care facility staff to resolve errors. Furthermore, in one embodiment the admitted patient dashboard indicates medication orders for newly admitted patients that can be fulfilled from an onsite medication dispensing unit 118.

In one embodiment, the techniques disclosed herein provide for acute care facility EMR/EHR connectivity for faster availability for new patient information. In one embodiment, an acute care facility EMR/EHR is provided with a list of non-acute care facilities that support receiving an electronic transfer of continuity of care data and having a pharmacy portal. The integration with acute care facility EMR/EHR systems provides the ability to update the acute care facilities with a current list of non-acute care facilities that are serviced by pharmacy systems implementing the techniques disclosed herein.

In one embodiment, the connectivity between acute care facilities and non-acute care facilities is provided by a healthcare information service provider, such as SureScripts™ HISP. The techniques provide for exchange of continuity of care data that include demographic data and medication orders for a patient.

In one embodiment, the continuity of care data is in the form of a continuity of care document electronically transferred to the non-acute care facility. The transfer of the continuity of care data is preferably performed in response to completing a discharge in the acute care facility's EMR/EHR system. That is, the continuity of care data is preferably transferred as soon as practical to the non-acute care facility after a decision has been made to discharge the patient and transfer them to a non-acute care facility. The electronic transfer of the continuity of care data may be performed extremely rapidly, subject to only comparatively minor delays required for electronic processing and secure electronic transport. In some embodiments, this permits the non-acute care facility to order any necessary medication before the patient arrives.

In one embodiment, the techniques provide for improved workflow in non-acute care facilities by, for example, organizing new patient information to reduce clinical transcription errors and streamline nurse workflow. In one embodiment, the workflow may include, for example, the EMR/EHR of the non-acute care facility determining medications to be ordered based on the received continuity of care data. The workflow may include receiving, at a pharmacy system, a message (e.g., an HL7 message) from the EMR/EHR system of the non-acute care facility upon creation of the resident chart. The message may include, for example, a request for medication order data from the pharmacy system. The workflow further includes parsing medication order data from the continuity of care document and providing orders in a ‘pending confirmation’ order status to the non-acute care facility's medication management system. The ‘pending confirmation’ orders may be reviewed, finalized, and submitted for fulfillment via the non-acute care facility's medication management system, such as via a pharmacy web portal. In some embodiments, the full continuity of care document may be transmitted to the non-acute care facility's EMR/EHR system.

In some embodiments, the techniques further include transmitting an admissions playbook to the non-acute care facility automatically (e.g., via fax). The admissions playbook may include an actionable worksheet using parsed data from the continuity of care document. The actionable worksheet allows nursing staff of the non-acute care facility to more efficiently submit or adjust new medication orders and reduces medication transcription errors. The admission playbook may further include additional sections of the continuity of care document as reference for the non-acute care facility, such as allergies, immunizations, discharge summaries, etc. In one embodiment, the techniques disclosed herein further provide the ability to query order details, date and time of data transfers, and other key performance indicators at multiple levels (e.g., account, non-acute care facility, patient, and order levels).

In one embodiment, the techniques disclosed herein provide alternative medication sourcing to suggest optimized orders based on availability or a formulary based on med-look-up algorithms. For example, for a new continuity of care document the techniques may include a search for onsite inventory within an onsite medication dispensing unit 118. Additionally, the search for available onsite medications may include searching for therapeutic alternatives to the medications in the discharge order based on inventory in the onsite medication dispensing unit 118 inventory. For example, many individual medications have therapeutic alternatives. Additionally, in one embodiment the search for available onsite medication may include a search for better formulary position opportunities, and include onsite medication dispensing unit 118 availability and potential alternatives as suggestions within the admissions playbook fax to provide staff at the non-acute care facility with an informed view of order options. Moreover, in one embodiment therapeutic equivalents or formulary equivalents may be used for other purposes, such as ordering therapeutic equivalents or formulary equivalents from the pharmacy.

In one embodiment techniques introduced herein improve operation of current admissions and medication delivery systems by providing expedited intake that improves pharmacy flow to expedite processing through acute care facility EMR/EHR discharge integration to parse patient demographics, clinical information, non-acute care facility details and billing information from discharge for new admits. In some embodiments, the techniques auto-create new patient profiles in the non-acute care facility EMR/EHR system and the pharmacy's medication management system based on the continuity of care document.

In the above description, for purposes of explanation, numerous specific details are set forth in order to provide a thorough understanding of the present disclosure. However, it should be understood that the technology described herein can be practiced without these specific details. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

To ease description, some elements of the system and/or the methods are referred to using the labels first, second, third, etc. These labels are intended to help to distinguish the elements but do not necessarily imply any particular order or ranking unless indicated otherwise.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms including “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Various implementations described herein may relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, including, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of an entirely hardware implementation, an entirely software implementation, or implementations containing both hardware and software elements. For instance, the technology may be implemented in software, which includes but is not limited to firmware, resident software, microcode, etc. Furthermore, the technology can take the form of a computer program object accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and Modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. As will be understood by those familiar with the art, the specification may be embodied in other specific forms without departing from the spirit or essential characteristics thereof. Likewise, the particular naming and division of the modules, routines, features, attributes, methodologies and other aspects are not mandatory or significant, and the mechanisms that implement the specification or its features may have different names, divisions and/or formats. Furthermore, the modules, routines, features, attributes, methodologies and other aspects of the disclosure can be implemented as software, hardware, firmware, or any combination of the foregoing. Also, wherever a component, an example of which is a module, of the specification is implemented as software, the component can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment.

What is claimed is:

1. A computer-implemented method comprising:
receiving, by a computing device, a notice of a new patient admission at a health care facility;
creating, by the computing device, a patient profile for a patient;
receiving, by the computing device, continuity of care data electronically communicated to the computing device in response to discharge of the patient from another health care facility;
reconciling, by the computing device, medication orders included in the continuity of care data; and
providing, by the computing device, instructions for fulfillment of the medication orders.

2. The method of claim 1, wherein the continuity of care data comprises a continuity of care document or extracts from a continuity of care document.

3. The method of claim 1, wherein the reconciling comprises determining medications available onsite at the health care facility relevant to fulfilling at least part of the medication orders.

4. The method of claim 3, wherein the instructions for fulfillment of the medication orders includes ordering medications of the medication orders not available onsite at the health care facility.

5. The method of claim 1, wherein the providing instructions comprises providing instructions for ordering at least one medication from a pharmacy.

6. The method of claim 1, further comprising automatically creating draft medication orders based at least in part on the medication orders in the continuity of care data.

7. The method of claim 1, further comprising generating a graphical user interface displaying the medication orders, an availability of onsite medications to fulfill at least a portion of the medication orders, and a status of any medications ordered to fulfill the medication orders.

8. The method of claim 7, wherein the graphical user interface comprises a pharmacy web portal with an admitted patient dashboard to order medications from a pharmacy and receive status notifications from the pharmacy.

9. The method of claim 1, wherein the health care facility is a non-acute care facility.

10. The method of claim 1, wherein the continuity of care data is electronically communicated utilizing security and transport management.

11. A computer program product comprising computer program instructions stored on a non-transitory storage medium, which when executed on a processor implements a method comprising:
receiving, at a non-acute care facility, continuity of care data electronically communicated in response to discharge of a patient from an acute care facility;
reconciling medication orders included in the continuity of care data; and
providing instructions for fulfillment of the medication orders.

12. The computer program product of claim 11, wherein the method further comprises generating a graphical user interface to reconcile medication orders and provide instructions for fulfillment of the medication orders.

13. A computer-implemented method, comprising:
generating continuity of care data for a patient selected to be discharged from an acute care facility, the continuity of care data including medication orders;
selecting a non-acute care facility for the patient to be transported to after discharge; and
electronically communicating, to the selected non-acute care facility, the continuity of care data in response to discharging the patient from the acute care facility.

14. The method of claim 13, further comprising:
generating information on therapeutic alternatives for the non-acute care facility for at least one medication of the medication orders; and
communicating the generated information to the non-acute care facility.

15. The method of claim 13, further comprising:
generating a summary of at least a portion of the continuity of care data; and
transferring the summary to the non-acute care facility.

16. The method of claim 13, wherein the continuity of care data comprises a continuity of care document or extracts from a continuity of care document.

17. The method of claim 13, further comprising providing the continuity of care data to a pharmacy servicing orders for the non-acute care facility.

18. The method of claim 13, further comprising utilizing the continuity of care data to reconcile medication orders for the patient at the non-acute care facility.

19. A system to expedite patient admissions and medication delivery, comprising:
an electronic medical or health records system of an acute care facility configured to generate continuity of care data and provide a secure electronic communication of the continuity of care data to a computing device of a non-acute care facility in response to discharging a patient from the acute care facility.
